(19) United States
(12) Patent Application Publication
(10) Pub. No.: US 2011/0044852 A1
(43) Pub. Date: Feb. 24, 2011

(54) IMPROVED DISINFESTATION METHOD

(76) Inventors: Robert Francis Ryan, Sans Souci (AU); Haris Krishna, Palmerston North (NZ)

Correspondence Address: The BOC Group, Inc., 575 MOUNTAIN AVENUE, MURRAY HILL, NJ, 07974-2082, US

(21) Appl. No.: 12/528,359

(22) PCT Filed: Feb. 25, 2008
(86) PCT No.: PCT/AU08/00248

§ 371 Date: Sep. 27, 2010

(30) Foreign Application Priority Data
Feb. 23, 2007 (NZ) 553418

(51) Int. Cl. A61L 9/015; A61L 2/20

(52) U.S. Cl. 422/32; 422/129

(57) ABSTRACT

A method and apparatus for disinfesting plant material in a chamber comprising a) a pesticide supply (2) for supplying a vaporising or aerosolised pesticide such as ethyl formate, b) a mixing device (3) for receiving and mixing the pesticide vapour or aerosol with a volume of diluent gas, and producing a first pesticide mixture, and c) a transfer system (4, 5) for introducing the first pesticide mixture into the chamber to form a second pesticide mixture. The chamber can be a greenhouse, silo or even a bag or box containing plant material.

### FIELD OF THE INVENTION

The invention relates to a method of disinfesting a chamber containing plant material, including but not limited to disinfesting plants growing in an enclosure and disinfesting packaged horticultural produce.

### BACKGROUND

Any discussion of the prior art throughout the specification should in no way be considered as an admission that such prior art is widely known or forms part of common general knowledge in the field.

Fresh produce obtained from horticultural crops forms an important part of the economies of most countries.

Such fresh produce is often grown in an enclosure such as a greenhouse or other nursery structures. While the environmental control within the enclosure is beneficial for crop growth, such controls also provide good conditions for the proliferation of unwanted organisms (including weeds, animals, arthropods, nematodes, bacterial and fungi) that can damage economic plants and their produce. It is typically necessary to take some additional steps to control these unwanted organisms and the most common technique is spraying or fogging with a pesticide or fumigant. Typically, unwanted organisms in such enclosures include insects, mites, spiders, nematodes, bacteria, fungi, fungal spores and viruses.

Irrespective of whether the produce is grown outdoors or in an enclosure such as a greenhouse, such fresh produce is often shipped to a destination far removed from where it is grown. Unfortunately, quarantine issues are compounded by the rapid expansion of world trade in fresh horticultural products and international passenger traffic. Despite any earlier attempts at pest control, or in cases where earlier pest control has not been carried out, unwanted organisms often infest plant produce before or during export. One problematic result is that the organisms can cause damage to produce through surface blemishes, destruction of edible flesh and spoilage from decay. This problem may result in serious economic impact on the grower of the produce. Moreover, the cost of fumigation is met by the importer, further reducing margins. A second problematic result is that if the export destination lacks effective controls, competing species or predators, the introduced organisms may become established and thrive, posing a threat to the stability of local agricultural industries and ecosystems. Postharvest treatments are therefore needed to disinfest fresh produce like whole plants, plant material including roots, bulbs, tubers, corms, leaves, flowers, seeds, callus tissue, nuts, grains, fruit, cuttings, root stock, scions, and harvested crops including roots, bulbs, tubers, corms, leaves, flowers, seeds, stems, callus tissue, nuts, grains, fruit, cuttings, root stock and scions, so the likelihood of the organism becoming established in the new location is minimised.

The most common method of destroying undesirable organisms on growing plants or travelling with plant produce is to disinfest the plants or plant produce with a pesticide or fumigant. Generally, plants or produce to be disinfested are located in an enclosure, for example the greenhouse in which plants are growing, the package in which the produce is placed or in a purpose built fumigation chamber. The enclosure is then filled with one or more chemicals toxic to the unwanted organisms. The disinfestation process involves creating a lethal concentration of the pesticide for a time sufficient to destroy the target pest. Ideally, the pesticide should be broad-spectrum and able to kill all unwanted organisms such as insects, mites, spiders, nematodes, bacteria, fungi, fungal spores, viruses and weed seeds. Additionally, the pesticide should leave either no residue or an inert residue, should be safe to handle, and should not adversely affect the plant or plant produce which is exposed to it.

Ethyl formate is a fumigant which is currently registered as a pesticide in Australia. It is considered to be a safer and more environmentally-friendly alternative to fumigants such as methyl bromide. It is classified as a low risk GRAS (generally recognised as safe) food additive and is easily hydrolysed to acceptable bi-products ethanol and formic acid.

Ethyl formate is a volatile compound. It has a boiling point of 54.3° C., a vapour pressure of 25.9 kPa at 20° C. and a low flash point. It is also highly flammable in its liquid state which may pose handling risks. However, the flammability of ethyl formate can be reduced by combining it with carbon dioxide (CO₂) or another diluent. It has been found that ethyl formate mixed with CO₂ not only retains its efficacy as a fumigant but may also have increased efficacy (see WO 03/061384).

Unfortunately, contact with relatively high concentrations of ethyl formate may cause damage to the plant or plant produce. For example, bananas and other fruit may show a patchy blackening of the peel at ethyl formate concentrations that may not be high enough to kill target pests travelling with the fruit.

It is an object of the present invention to overcome or ameliorate at least one of the disadvantages of the prior art, or to provide a useful alternative.

According to a first aspect, the invention provides a method of disinfecting plant material in a chamber, the method comprising
a) vaporising or aerosolising a pesticide,
b) mixing the vapour or aerosol with a volume of a diluent gas to create a first pesticide mixture, and
c) introducing the first pesticide mixture into the chamber to form a second pesticide mixture.

According to a second aspect, the invention provides a method of disinfecting plant material in a chamber, the method comprising
a) aerosolising a pesticide, to form droplets or particles of from about 0.5 to about 30 μm in diameter,
b) mixing the aerosol with a volume of a diluent gas to create a first pesticide mixture,
c) introducing the first pesticide mixture into the chamber, and
d) circulating the first pesticide mixture throughout the majority of the chamber to form a substantially homogenous second pesticide mixture.

According to a third aspect, the invention provides a method of disinfecting plant material in a chamber, the method comprising
a) vaporising pesticide,
b) mixing the vapour with a volume of a diluent gas to create a first pesticide mixture,
c) introducing the first pesticide mixture into the chamber, and
d) circulating the first pesticide mixture throughout the majority of the chamber to form a substantially homogenous second pesticide mixture.

In a fourth aspect the invention relates to a method of disinfesting plant material comprising
a) vaporising or aerosolising a pesticide,
b) mixing the vapour or aerosol with a volume of a diluent gas to create a first pesticide mixture,
c) reducing the pressure in the chamber to below atmospheric pressure, and
d) introducing the first pesticide mixture into the chamber to produce a second pesticide mixture.

In one embodiment the first pesticide mixture is circulated through at least about 50% of the gas volume of the chamber, preferably at least about 60%, at least about 70%, at least about 80%, at least about 90% or about 100% of the gas volume of the chamber.

In one embodiment the gas in the chamber is used as the diluent gas and is mixed with the vapour or aerosol to create the first pesticide mixture. Preferably the gas in the chamber is recycles until a substantially homogenous second pesticide mixture is formed in the chamber.

According to a fifth aspect, the invention provides an apparatus for disinfesting plant material in a chamber comprising
a) a pesticide supply for supplying a vaporising or aerosolised pesticide,
b) a mixing device for receiving and mixing the pesticide vapour or aerosol with a volume of diluent gas, and producing a first pesticide mixture, and
a transfer system for introducing the first pesticide mixture into the chamber to form a second pesticide mixture.

In one embodiment the first pesticide mixture is introduced into the chamber using a network of ducting.

In one embodiment the ducting comprises flexible aluminium or plastic tubing. The desired diameter of the ducting is dependent on the size of the chamber. Ducting is readily available in a range of diameters. Preferably the ducting is at least about 100 mm, at least about 150 mm, at least about 200 mm, at least about 250 mm, at least about 300 mm, at least about 350 mm, at least about 400 mm, at least about 450 mm, or at least about 500 mm in diameter. When the chamber is a larger chamber such as a greenhouse or other nursery structure, the ducting is preferably between about 300 to about 500 mm in diameter, more preferably about 400 mm in diameter. Preferably the ducting comprises an expanded volume of layflat plastic tubing.

In one embodiment the ducting capture aerosol droplets or particles in the first pesticide mixture that are not from about 0.5 to about 30 μm in diameter or that have not completely vaporised. Preferably, the ducting captures aerosol droplets or particles that are not from about 1 to about 25 μm in diameter, more preferably about 2 to about 20 μm in diameter.

In one embodiment the ducting comprises a plurality of holes spaced along the length of the ducting. In one embodiment the holes are located in the upper portion of the ducting.

Preferably the holes are located along either side of the horizontal axis of the ducting. In one embodiment the holes on one side of the ducting are offset in relation to the holes on the opposite side of the ducting.

The number and diameter of the holes is dependent on the relative diameter of the ducting. In one embodiment the holes are at least about 10 mm, at least about 15 mm, at least about 20 mm, at least about 25 mm, at least about 30 mm, at least about 35 mm, at least about 40 mm, at least about 45 mm, or at least about 50 mm in diameter. When the ducting is between about 300 to about 500 mm in diameter, the holes are preferably between about 30 mm to about 50 mm in diameter. In ducting of about 400 mm in diameter the holes are preferably between about 35 mm to about 40 mm in diameter, more preferably about 38 mm in diameter.

In layflat plastic tubing inflated to about 400 mm in diameter it was found that up to 110 holes of about 38 mm in diameter and offset from each other along the horizontal axis of the tubing were the threshold at which optimum dynamic and static pressures in the tubing could be maintained while allowing the even introduction and distribution of the aerosol.

The following embodiments may relate to any of the above aspects of the invention.

In one embodiment the method further comprises maintaining the second pesticide mixture in the chamber for a time sufficient to disinfest the plant material. In another embodiment the method further comprises sealing the chamber, optionally hermetically sealing the chamber.

In one embodiment the plant produce is selected from the group comprising fruits, vegetables, grains, flowers, propagative material such as seeds or cuttings and other nursery stock. Preferably, the plant produce comprises fruit. More preferably, the plant produce comprises fruit selected from the group comprising bananas, pineapples, apples, kiwifruit, avocados, citrus, feijoas, persimmons or summerfruit but not limited thereto.

In one embodiment the second pesticide mixture is toxic to insects, mites, spiders, nematodes, bacteria, fungi and their spores and viruses.

In one embodiment the diluent gas used to form the first pesticide mixture is air, carbon dioxide or nitrogen.

In one embodiment the stored form of the pesticide includes a carrier, such as carbon dioxide. In one embodiment the method comprises providing a liquid pesticide concentrate comprising a pesticide dissolves in liquid CO₂. This pesticide concentrate is stored under pressure until needed. In use the liquid carrier phase vaporises forming an aerosolised pesticide. In one embodiment the liquid pesticide also vaporises.

In one embodiment the first pesticide mixture comprises at least one vaporised or aerosolised pesticide and air. In one embodiment the pesticide concentrate is vaporised or aerosolised and mixed with air to form a substantially homogenous mixture.

In one embodiment the pesticide is selected from the group comprising insect grown regulators, botanicals, pyrethrins derivatives, synthetic pyrethroids, chlorinated aryl hydrocarbons and DDT relatives (diphenyl aliphatics), avermectins, carbamates, organophosphates, chloronicotinyl, pyridazinone, spinosyns, sulfonates, benzoylurea, nitriles, triazoles, morpholine, dicarboxidie, and mixtures thereof.

In one embodiment the first pesticide mixture comprises at least one vaporised pesticide and air. In one embodiment the pesticide is heated before being mixed with air to form the pesticide mixture. In one embodiment the first pesticide mixture is heated. Preferably any heating step comprises heating to at least about 40, 45, 50, 55, 60, 65, or 70° C. and useful ranges may be selected between any of these values (for example, from about 45 to about 65° C.).

In one embodiment the pesticide is a fumigant selected from the group comprising acetaldehyde, azobenzene, carbon disulphide, carbon tetrachloride, carbonyl sulphide, carvone, chloroform, chloropicrin, cyanogens (including but not limited to acrylonitrile, hydrogen cyanide and methyl isothiocyanate), dichloronitroethane, 1,3-dichloropropene, dichlorvos (dimethyl 2,2-dichlorovinyl phosphate; DDVP), essential oils (including but not limited to essential oils from rosemary, thyme, palmarosa and basil), ethyl acetate, ethylene chlorobromide, ethylene dibromide, ethylene dichloride, ethylene oxide, ethyl formate, methyl allyl chloride, methyl bromide, methyl chloroform (1,1,1-trichloroethane), methylene chloride, methyl formate, methyl iodide, nicotine, phosphine, propylene dichloride, propylene oxide, sulphur dioxide, sulphuryl fluoride and mixtures thereof.

In one preferred embodiment the pesticide is selected from the group comprising ethyl formate, hydrogen cyanide, methyl bromide, phosphine and mixtures thereof. In another preferred embodiment the pesticide is ethyl formate.

In a preferred embodiment the pesticide is VAPORMATE® (a pesticide manufactured and marketed by BOC Limited) which comprises liquid ethyl formate and liquid CO₂ stored under pressure at approximately a 1:6 ratio by weight. When this material is released it forms a 1:11 ratio of ethyl formate to CO₂ by volume.

In one embodiment the first pesticide mixture comprises at least about 5, 10, 15, 20, 25, 30, 35, 40, 45, 50, 55, 60, 65, 70, 75, 80, 85, 90 or 95% air by volume and useful ranges may be selected between any of these values (for example, from about 5 to about 95%, from about 20 to about 80%, from about 30 to about 80%, from about 40 to about 80%, from about 50 to about 80%, from about 60 to about 80% and from about 70 to about 80%). Preferably the first pesticide mixture comprises from about 50 to about 80% air by volume.

In one embodiment the first pesticide mixture comprises at least about 1, 2, 3, 4, 5, 6, 7, 8, 9, 10, 12, 15, 18, 20, 22, 25, 28, 30, 32, 35, 38, 40, 42, 45, 48 or 50% of one or more pesticides by volume and useful ranges may be selected between any of these values (for example, from about 1 to about 40%, from about 1 to about 20%, from about 3 to about 7% and from about 4 to about 5%). Preferably the first pesticide mixture comprises from about 10 to 45% of one or more pesticides by volume.

In one embodiment the first pesticide mixture comprises about 1 to about 8% ethyl formate, about 11 to about 88% CO₂ and about 4 to about 88% air by volume.

In one embodiment the first pesticide mixture comprises about 2-2.5% ethyl formate, about 22.5% CO₂ and about 75% air by volume.

In one embodiment the first pesticide mixture comprises at least about 5, 10, 15, 20, 25, 30, 35, 40, 45, 50, 55, 60, 65, 70, 75, 80, 85, 90 or 95% pesticide concentrate (active agent(s) plus carrier) by volume and useful ranges may be selected between any of these values (for example, from about 5 to about 95%, from about 20 to about 30%, from about 20 to about 40%, from about 20 to about 50%, from about 20 to about 60%, from about 20 to about 70% and from about 20 to about 80%).

In one embodiment the pesticide concentrate comprises at least about 1, 5, 10, 15, 20, 25, 30, 35, 40, 45 or 50% pesticide by volume and useful ranges may be selected between any of these values (for example, from about 1 to about 50%, from about 10 to about 40%, from about 10 to about 30% and from about 15 to about 25%). Preferably the pesticide concentrate comprises about 10 to about 35% pesticide by volume, more preferably about 10 to about 15% pesticide by volume.

In one embodiment the pesticide concentrate comprises at least about 50, 55, 60, 65, 70, 75, 80, 85, 90, 95 or 99% CO₂ by volume and useful ranges may be selected between any of these values (for example, from about 50 to about 99%, from about 60 to about 90%, from about 70 to about 90% and from about 75 to about 85%). Preferably the pesticide concentrate comprises about 65 to 90% CO₂, more preferably about 85 to about 90% CO₂.

In one embodiment the chamber is a greenhouse, shipping container, rail car, warehouse, closed room or tent. In one embodiment the plant produce is packaged before being placed in the chamber. In such an embodiment, the chamber is filled to a pressure no more than slightly above atmospheric pressure.

In one embodiment the chamber is a film, bag or box, or a combination thereof. In one embodiment the chamber is a semi-permeable film that allows controlled exchange of gases. Such packaging is suitable for transporting fresh plant produce. In another embodiment the chamber is a hermetic package. In one embodiment the chamber is hermetically sealed. In a case where the chamber is a bag or box etc, it may be pressurised to above atmospheric pressure, ie 1-3 bar.

In one embodiment the second pesticide mixture is maintained in the chamber for at least about 2, 4, 8, 10, 12, 24, 32 or 48 hours and useful ranges may be selected between any of these values (for example, from about 2 to about 48 hours). Efficacious application rates for different pesticides and different target pests are well described in the literature. The concentration of pesticide(s) in the second pesticide mixture can thus be customised to correspond with the type of chamber used, the type of plant material to be treated and the identity of the pest.

In one embodiment the method precedes or follows treatment with an additional pesticide.

In one embodiment the additional pesticide is selected from the group comprising insect growth regulators, botanicals, pyrethrins derivatives, synthetic pyrethroids, chlorinated aryl hydrocarbons and DDT relatives (diphenyl aliphatics), avermectins, carbamates, organophosphates, chloronicotinyl, pyridazinone, spinosyns, sulfonates, benzoylurea, nitriles, triazoles, morpholine, dicarboxidie, and mixtures thereof.

In one embodiment the additional pesticide is a fumigant selected from the group comprising acetaldehyde, azobenzene, carbon disulphide, carbon tetrachloride, carbonyl sulphide, carvone, chloroform, chloropicrin, cyanogens (including but not limited to acrylonitrile, hydrogen cyanide and methyl isothiocyanate), dichloronitroethane, 1,3-dichloropropene, dichlorvos (dimethyl 2,2-dichlorovinyl phosphate; DDVP), essential oils (including but not limited to essential oils from rosemary, thyme, palmarosa and basil), ethyl acetate, ethylene chlorobromide, ethylene dibromide, ethylene dichloride, ethylene oxide, ethyl formate, methyl allyl chloride, methyl bromide, methyl chloroform (1,1,1-trichloroethane), methylene chloride, methyl formate, methyl iodide, nicotine, phosphine, propylene dichloride, propylene oxide, sulphur dioxide, sulphuryl fluoride and mixtures thereof.

In one preferred embodiment the additional pesticide is selected from the group comprising ethyl formate, hydrogen cyanide, methyl bromide, phosphine and mixtures thereof. In another preferred embodiment the additional pesticide is ethyl formate.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application ion a similar manner.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE DRAWINGS

FIG. 1 is a schematic of a device for forming a pesticide mixture and introducing it into a chamber, preferably a greenhouse.

FIG. 2 is a schematic of the mixing section of the system.

FIG. 3 is a schematic of a delivery tube of the system.

FIG. 4 is a cross-section of a joint in a delivery tube in a system for introducing a pesticide mixture into a chamber.

FIG. 5 is an end view of the joint of FIG. 4.

### DETAILED DESCRIPTION OF THE INVENTION

The present invention provides a method of disinfesting plant material. In one aspect the present invention relates to a method of disinfesting plant material in a chamber, the method comprising
a) providing a chamber containing plant material,
b) forming a vapour or an aerosol of pesticide,
c) mixing the vapour or aerosol with a volume of a gas to create a first pesticide mixture, and
d) introducing the first pesticide mixture into the chamber to create a second pesticide mixture.
In one embodiment the method for further comprises maintaining the second pesticide mixture in the chamber for a time sufficient to disinfest the plant material. In another embodiment the method further comprises sealing the chamber.

### 1. DEFINITIONS

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

The terms “disinfest”, “disinfesting” and “disinfestation” as used herein refer to killing unwanted organisms (including plants, animals, arthropods, nematodes, bacterial and fungi) that can damage the plants or the resulting produce, but do not extend to removing the remains of killed unwanted organisms from the chamber.

The term “plant material” is intended to include whole plants, roots, bulbs, tubers, corms, leaves, flowers, seeds, stems, callus tissue, nuts, grains, fruit, cuttings, root stock, scions, and harvested plant produce.

The term “plant produce” refers to any produce obtained from a plant or plant material including but not limited to fruit, vegetables, roots, bulbs, tubers, corms, leaves, flowers, seeds, stems, callus tissue, nuts; crop products such as cereals, rice, wheat, corn and beans; propagative material such as root stock, scions, seeds or cuttings; and other plant material such as wood (including sawn timber and wood products), bark or cut flowers.

In one embodiment the plant produce comprises fruit including but not limited to berries (including grapes, dates, avocado, persimmons, eggplant, guava, and chili peppers), drupes (including coffee, coconut, mango, olive, apricot, cherry, peach, nectarine and plum), citrus (including grapefruit, lemons, limes, mandarins, oranges and tangerines), false berries (including bananas, blueberries, cranberries, gooseberries, watermelon, cucumbers, squash, pumpkins, and currants such as blackcurrants and redcurrants), pome fruit (including apples, pears and quince), strawberries, pineapples and kiwifruit.

In one embodiment the plant produce comprises vegetables including but not limited to artichoke, rocket, asparagus, avocado, beans, peas, black-eyed peas, black-eyed beans, chickpeas, garbanzo beans, lentils, limas, mung beans, soybeans, mangetout, snow peas, broccoflower, broccoli, Brussels sprouts, cabbage, cauliflower, celery, chard, collard greens, corn, sweetcorn, maize, eggplant, aubergine, fennel, lemon grass, lettuce, okra, chives, garlic, leek, onion, shallot, parsley, peppers, capsicum, jalapeno, paprika, Tabasco, cayenne pepper, radish, rhubarb, root vegetables, beetroot, carrot, ginger, parsnip, radish, swede, turnip, wasabi, white radish, spinach, spring greens, squashes, chayote, courgette, zucchini, cucumber, pumpkin, spaghetti squash, winter melon, tubers, manioc, potato, sweet potato, kumara, taro, yam, water chestnut, watercress, water mimosa, and waternut. Preferably the vegetables are onions or lettuce.

In one embodiment, the plant material is a growing plant selected from plants that produce or comprise one of plant produce items listed above. It has been noted that unlike previous systems, the proposed methods and apparatus are particularly suited to application on growing plants in greenhouses. Such greenhouses generally can only withstand low pressures, ie slightly above atmospheric.

The term “hermetic package” refers to a package made from a packaging material that is substantially impervious to gas. A hermetic package may be any container, box or bag capable of forming a substantially closed system.

### 2. TREATMENT CHAMBERS

The disinfestation methods of the invention may be carried out on plants, including growing plants, or plant produce that is located in a chamber including an enclosure such as a greenhouse or other nursery structures, a fumigation chamber (a dedicated chamber for the elimination of pests on plant produce or packaged plant produce or both) or a package (whether transitory or intended for final sale).

The chamber may be of any shape. Preferred chambers include but are not limited to enclosures (such as greenhouses—glasshouses, hothouses or other nursery structures —, shipping containers, rail cars, warehouses, closed rooms, tents and the like. Preferably the chamber is capable of forming a substantially closed system. Fumigation chambers include portable flexible chambers such as Boracure® (Boracure Auckland Ltd, Auckland, New Zealand) and GrainPro® (GrainPro Inc, Concord Mass., USA) made of non-permeable fumigation plastic.

In one embodiment the plant material may be present in the chamber in one or more bags, boxes, packages or other containers. For the packaging of fresh plant produce the package or container must be gas permeable or must be altered to be so, for example by opening it or cutting holes in it, to allow for gas exchange.

Alternatively, the methods of the invention can be applied directly to a container holding the plant produce, such as packaging material including boxes, bags, crates and containers. Preferred packaging includes hermetic packages but a package useful herein may be any bag, box, package or other container capable of carrying plant produce.

### 3. PESTICIDES

A pesticide for use in the methods of the invention may be any pesticide or blend of pesticides, whether solid or liquid, that is capable of being aerosolised and/or vaporised.

In one embodiment, the pesticides for use in the methods of the invention may be any pesticide or blend of pesticides that is dissolvable in liquid CO₂.

Pesticides for use in the invention as aerosols include but are not limited to insect growth regulators, botanicals, pyrethrins derivatives, synthetic pyrethroids, chlorinated aryl hydrocarbons and DDT relatives (diphenyl aliphatics), avermectins, carbamates, organophosphates, chloronicotinyl, pyridazinone, spinosyns, sulfonates, benzoylurea, nitriles, triazoles, morpholine, dicarboxidie, and blends thereof.

Pesticides for use in the invention that are useful as fumigants include but are not limited to acetaldehyde, azobenzene, carbon disulphide, carbon tetrachloride, carbonyl sulphide, cavone, chloroform, chloropicrin, cyanogens (including but not limited to acrylonitrile, hydrogen cyanide and methyl isothiocyanate), dichloronitroethane, 1,3-dichloropropene, dichlorvos (dimethyl 2,2-dichlorovinyl phosphate; DDVP), essential oils (including but not limited to essential oils from rosemary, thyme, palmarosa and basil), ethyl acetate, ethylene chlorobromide, ethylene dibromide, ethylene dichloride, ethylene oxide, ethyl formate, methyl allyl chloride, methyl bromide, methyl chloroform (1,1,1-trichloroethane), methylene chloride, methyl formate, methyl iodide, nicotine, phosphine, propylene dichloride, propylene oxide, sulphur dioxide, sulphuryl fluoride and blends thereof.

In one embodiment the pesticide is toxic to unwanted organisms including one or more insects, mites, spiders, nematodes, bacteria, fungi, fungal spores and viruses, or any combination of two or more thereof.

Preferably, the pesticide is toxic one or more pests of the families Lepidoptera (light brown apple moth, codling moth, warehouse moth, dart moths, blister moth, leaf miner, cutworms, corn borer, bollworm, potato moth, armyworm, clothes moth, loopers), Acari (spiders including black widow spiders, flour mites, grain mites, rust mites, ticks, tea mite, tetranychid mites, eriophyid mites, mange mites, citrus red mite, red spider mites, rust mists, two-spotted mites, bulb mites, tarsonemid mites, spider mites), Homoptera (greenhouse whitefly, glasshouse potato aphid, rose aphid leafhoppers, scale insects, mealybugs), Diptera (mosquitoes, root flies, fruit flies, midges, house flies, mushroom cecid, leave miners, mushroom sciarid flies, cereal fly, carrot fly, fungus gnats), Coleoptera (mealworms, furniture beetles, carpet beetles, house longhorn, beetles, cereal leaf beetle, seed weevil, grain beetles, flour beetles, rice beetle, pea and bean weevil, maize weevil, rust red flour beetle, confused flour beetle), Thysanoptera (thrips including greenhouse thrips, western flower thrips, onion thrips, rose thrips) or any combination of any two or more thereof.

Other target pests include unwanted species of ants, aphids, bees, beetles, bugs, butterflies, flies, midges, mites, moths, sawflies, scales, thrips, wasps, weevils. Other target pests include those identified as New Zealand pest species at http://mafuwsp6.maf.govt.nz/uor/searchframe.htm.

In one embodiment the pesticide is ethyl formate.

In one embodiment the method comprises providing a pesticide concentrate comprising one or more liquid pesticides dissolved in liquid CO₂. The pesticide concentrate is stored under pressure until needed. In one embodiment the pesticide concentrate is vaporised or aerosolised and mixed with air to form a substantially homogenous mixture.

In one embodiment the pesticide concentrate is vaporised by heating before being mixed with air to form the first pesticide mixture. In one embodiment the first pesticide mixture is heated. Preferably any heating step comprises heating the pesticide concentrate or first pesticide mixture to at least about 40, 45, 50, 55, 60, 65 or 70° C. and useful ranges may be selected between any of these values (for example, from about 45 to about 65° C.).

In one embodiment the first pesticide mixture comprises at least about 5, 10, 15, 20, 25, 30, 35, 40, 45, 50, 55, 60, 65, 70, 75, 80, 85, 90 or 95% air by volume and useful ranges may be selected between any of these values (for example, from about 5 to about 95%, from about 20 to about 80%, from about 30 to about 80%, from about 40 to about 80%, from about 50 to about 80%, from about 60 to about 80% and from about 70 to about 80%).

In one embodiment the first pesticide mixture comprises at least about 1, 2, 3, 4, 5, 6, 7, 8, 9, 10, 12, 15, 18, 20, 22, 25, 28, 30, 32, 35, 38, 40, 42, 45, 48 or 50% of one or more pesticides by volume and useful ranges may be selected between any of these values (for example, from about 1 to about 40%, from about 1 to about 20%, from about 3 to about 7% and from about 4 to about 5%). Preferably the first pesticide mixture comprises from about 10 to 45% of one or more pesticides by volume.

In one embodiment the first pesticide mixture comprises about 3 to about 15% by volume ethyl formate, about 18 to about 27% by volume CO₂ and about 70 to about 75% by volume air.

In one embodiment the first pesticide mixture comprises about 2-2.5% ethyl formate, about 22.5% CO₂ and about 75% air by volume.

In one embodiment the first pesticide mixture comprises at least about 5, 10, 15, 20, 25, 30, 35, 40, 45, 50, 55, 60, 65, 70, 75, 80, 85, 90 or 95% pesticide concentrate by volume and useful ranges may be selected between any of these values (for example, from about 5 to about 95%, from about 20 to about 30%, from about 20 to about 40%, from about 20 to about 50%, from about 20 to about 60%, from about 20 to about 70% and from about 20 to about 80%).

In one embodiment the pesticide concentrate comprises at least about 1, 5, 10, 15, 20, 25, 30, 35, 40, 45 or 50% of one or more pesticides by volume and useful ranges may be selected between any of these values (for example, from about 1 to about 50%, from about 10 to about 40%, from about 10 to about 30% and from about 15 to about 25%). Preferably the pesticide concentrate comprises about 10 to about 35% pesticide by volume, more preferably about 10 to about 15% pesticide by volume.

In one embodiment the pesticide concentrate comprises at least about 50, 55, 60, 65, 70, 75, 80, 85, 90, 95 or 99% CO₂ by volume and useful ranges may be selected between any of these values (for example, from about 50 to about 99%, from about 60 to about 90%, from about 70 to about 90% and from about 75 to about 85%). Preferably the pesticide concentrate comprises about 65 to 90% CO₂, more preferably about 85 to about 90% CO₂.

In one embodiment the method conducted at about 15° C. to about 30° C.

In one embodiment the second pesticide mixture is maintained in the chamber for at least about 2, 4, 8, 10, 12, 24, 32 or 48 hours and useful ranges may be selected between any of these values (for example, from about 2 to about 48 hours). Efficacious application rates for different pesticides and different target pests are well described in the literature. The concentration of pesticide(s) in the second pesticide mixture can thus be customised to correspond with the type of chamber used, the type of plant material to be treated and the identity of the pest.

### 4. DISINFESTATION OF LARGER CHAMBERS

Methods of the invention may be employed using large chambers including enclosures such as greenhouse or other nursery structures containing plant material.

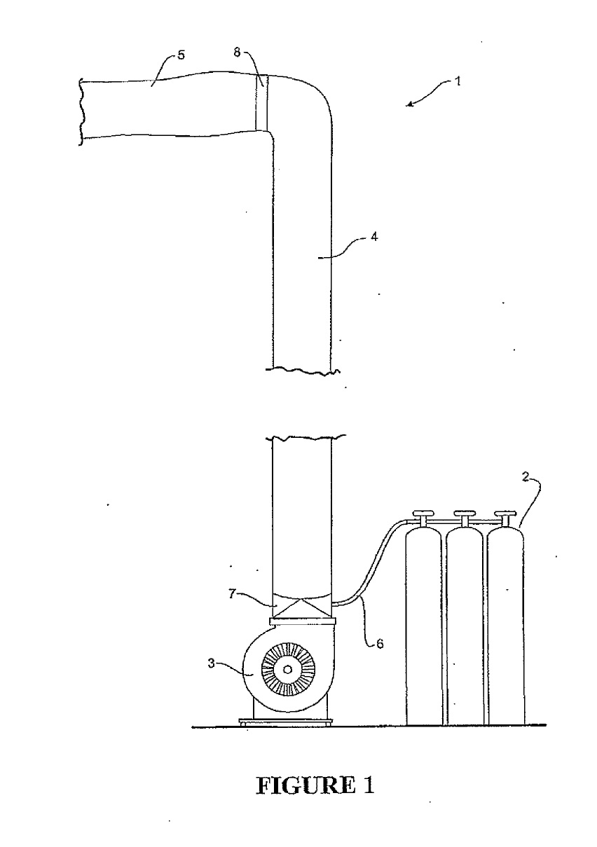

FIGS. 1 to 4 show the components of a system that may be used to form and introduce and aerosolised pesticide mixture into a greenhouse, for example. It should be understood that this system is only one way of introducing a pesticide mixture into a greenhouse and other known methods may include known fans and ventilation ducting systems.

FIG. 1 depicts a device for forming and introducing an aerosolised pesticide mixture into a greenhouse. The device 1 comprises a pesticide source 2, a fan 3, a fan hood 4 and a network of ducting 5.

The pesticide source 2 will typically comprise a liquid pesticide and a carrier under pressure. However, other pesticide may comprise a liquid or powdered solid source provided the source is able to be aerosolised.

In the embodiment depicted in FIG. 1, the fan hood 4 comprises 400 mm diameter tube connected to the air outlet of the fan 3. In use, the fan 3 forces air into the hood 4 to form an air steam into which the pesticide is aerosolised using a spray nozzle, forming a first pesticide mixture when is then forced through the hood and into the ducting network 5.

The ducting network 5 preferably comprises a network of layflat plastic tubing. The plastic tubing will be connected to the hood 4 at the junction point 8 and is inflated into an operating state by the fan 3. Therefore, the fan 3 not only acts to form the first pesticide mixture and to force the mixture into the ducting network, it acts to inflate the ducting network into the operating inflated state comprising a plastic tube having a diameter approximately equivalent to the diameter of the hood 3.

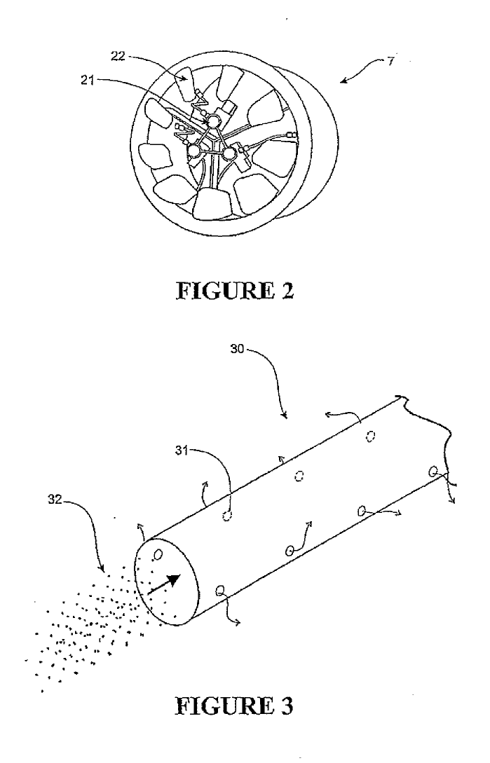

The pesticide source 2 is delivered into the fan hood 4 by a connecting hose 6. Connecting hose 6 engages a mixing unit 7. Mixing unit 7 is shown in more detail in FIG. 2. Mixing unit 7 may be positioned in the fan hood 3 anywhere between position 7 (as shown in FIG. 1) and position 8.

FIG. 2 depicts a preferred mixing unit 7. Connecting hose 6 (not shown) delivers pesticide to a plurality of nozzles 21. Nozzles 21 are adapted to aerosolise the pesticide into the air flow from fan 3 (not shown). Mixing unit 7 also comprises vanes 22 that create turbulence n the air flow from fan 3. Air passing through the mixing unit is combined with pesticide from nozzles 21 and the action of vanes 22 mixes the pesticide and air from inside the chamber to form the first pesticide mixture.

FIG. 3 depicts a section of the ducting network 5 comprising inflated lay-flat tubing 30 that comprises holes 31 spaced and offset along either side of the horizontal axis of the tubing. In preferred embodiments there are no holes in a portion that would allow liquid collected in the tubing to exit the tubing; for example, there are no holes in the bottom portion of the tubing below the horizontal axis of the tubing. This allows the ducting to capture aerosol droplets or particles in the first pesticide mixture that are not from about 0.5 to about 30 μm in diameter or that have not completely vaporised. Preferably the ducting captures aerosol droplets or particles that are not from about 1 to about 25 μm in diameter, more preferably that are not from about 2 to about 20 μm in diameter. This configuration minimises the introduction of large aerosol droplets or particles of high pesticide and/or solvent concentration being introduced into the chamber, minimising the damage to plant material. Any condensate captured in the ducting can be removed at a later date or left to vaporise over time.

The first pesticide mixture 32 is blown into and through the ducting network 5. For a given section of tubing 30, the first pesticide mixture exits the tubing through holes 31. The ducting network is arranged so that first pesticide mixture 32 exiting holes 31 is substantially evenly introduced into the air present in the upper region of the chamber. The system is run and the air inside the chamber recycles until the pesticide mixture has diffused into the lower region of the chamber, forming a substantially homogenous second pesticide mixture throughout the majority of the chamber. The system can then be shut down to allow the aerosolised pesticide to settle onto the plant material. This distribution allows for a substantially homogenous distribution of the pesticide mixture throughout the majority of the chamber, minimising areas of high and low pesticide concentration that damage plant material or result in poor efficacy, respectively.

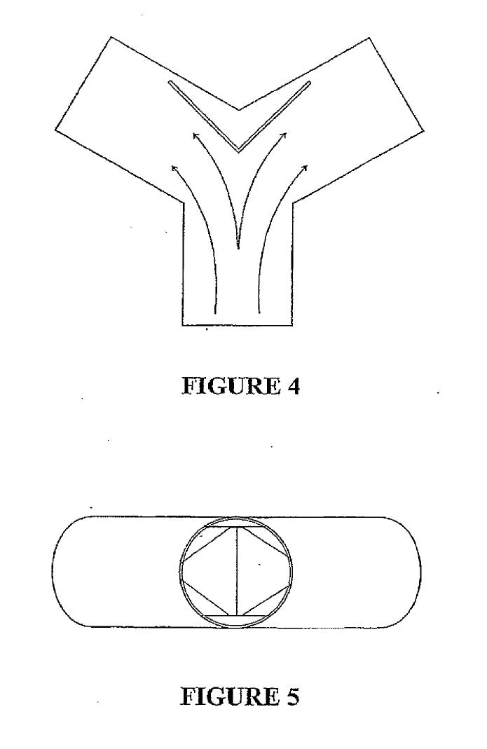

FIG. 4 shows a cross-section of a joint located in the hood 4 thought which the first pesticide mixture can be divided before entering the ducting network 5. Arrows indicate the flow of the aerosol droplets or particles as the first pesticide mixture exits the hood and enters the ducting network. FIG. 5 is an end view of the joint of FIG. 4.

The greenhouse pesticide application systems typically utilise a network of ¼ inch aluminium pipes located throughout the upper region of the greenhouse, exiting in clusters of nozzles which aerosolise the pesticide concentrate into the greenhouse. It was noted in Example 5 that these systems result in severe crop damage to plants within the vicinity of the nozzles after each spray, due to limited or poor aerosol production. The limited aerosol production also affected the efficacy of the spraying, resulting in variable poor to below optimal control of pests.

The applicants have discovered that premixing the aerosolised pesticide with a diluent carrier gas such as carbon dioxide and air before application into the greenhouse allowed for better aerosolising of the pesticide and distribution of the aerosol throughout the greenhouse, resulting in high levels of efficacy with no observable damage to plant material.

### 5. DISINFESTATION OF SMALLER CHAMBERS

Methods of the invention may be employed using smaller chambers including shipping containers, rail cars, warehouses, closed rooms, tents, fumigation chambers or packaging (whether transitory or intended for final sale).

In one embodiment the pesticide mixture is simply blown into the chamber using a conventional system for pumping gases, such as a fan.

In another embodiment the chamber is partially or fully evacuated to reduce the air pressure below atmospheric pressure prior to introduction of the pesticide mixture. In such embodiments the pesticide concentration in the first and second pesticide mixtures will be substantially the same. Evacuation can be achieved using any means known in the art, for example, by using a commercial vacuum cleaner. A non-return valve can be used to hold the partial or full vacuum until the pesticide mixture is introduced. Introducing the pesticide mixture into a partial air-free space allows it to be distributed more evenly.

In a preferred embodiment the pesticide is the fumigant ethyl formate. Ethyl formate combined with carbon dioxide is commercially available as VAPORMATE® (BOC Ltd) and is described in WO 03/061384 which is incorporated by reference.

VAPORMATE® comprises 16.7% by volume ethyl formate in liquid carbon dioxide. It is available in cylinders containing about 6.0 and 31 kg product. The operating pressure from the cylinder is about 5500 kPa. The VAPORMATE® is dispenses as small, volatile ethyl formate aerosol particles (approx 2-20 μm). These particles vaporise in the ambient air to form a vapour.

VAPORMATE® is effective at killing a large range of pests of quarantine interest. However, even when applies to a partially evacuated space, VAPORMATE® does not distribute evenly. This can be seen in Example 1 where aerosolised VAPORMATE® was applied to packaged bananas and resulted in peel injury.

VAPORMATE® was very effective at killing both mealybugs and mites when applied at concentrations between 20 and 40 g/m³ of active ingredient, ie ethyl formate. VAPORMATE® was found to have high efficacy (99-100% mortality) when compared to the other fumigants OFG-1 (0.5% pyrethrins, 4.5% ethanol) and sulphur dioxide gas. However, at all the VAPORMATE® concentrations tested, many of the bananas developed black patches on their peel.

In Example 2 the range of VAPORMATE® concentrations was widened to between 10 and 50 g/m³ of active ingredient, ie ethyl formate. Even at the lowest concentration tested (10 g/m³ ethyl formate), unacceptable peel damage occurred.

It was noted in Example 2 that pests closer to the point of application were more severely affected than those placed further away. This suggests that, when applies as an aerosol, much of the product was deposited as a liquid close to the point of entry, gradually vaporising to give a higher concentration of fumigant near the entry point. Consequently, the killing rate was not satisfactory for pests located far from the entry point, including those located deep in the volume of produce.

This is also thought to contribute to the peel damage observed on the bananas. Disinfestation with the poorly mixed and distributed ethyl formate aerosol exposes the bananas to VAPORMATE® at higher concentrations in small areas. Fruit with this blackening damage is not considered to be acceptable quality for sale. Consequently, although VAPORMATE® is a potential safe fumigant for the disinfestation of packaged bananas, peel injury arising from aerosol application can hinder its use.

Applying ethyl formate as a vapour enhances the efficacy of disinfestation, as can be seen in Example 2.

Vaporisation can be carried out by any means known in the art, including heating the fumigant to above its boiling point. For example, a vaporiser can be constructed using a coil of ¼ aluminium pipe heated by an element. The element heats the coiled aluminium pipe, through which the fumigant travels. The temperature needs to be maintained at higher than 54.3° C. for vaporisation of ethyl formate. To achieve complete vaporisation the fumigant can be released into the delivery pipe using a restrictor to slow down the flow rate.

In one embodiment the temperature will be maintained at between about 50° C. to about 70° C.

Different fumigants particularly those which are liquid at STP (Standard Temperature Pressure) require heating at different temperatures to achieve vaporisation, depending on their boiling points. If the fumigant is a gas at STP it will not need to be vaporised before mixing with the other gaseous components. It is to be understood that a skilled worker will be able, without undue experimentation and with regard to that skill and this disclosure, to ascertain the conditions needed to vaporise the fumigant.

As can be seen in Example 2, vaporisation of the ethyl formate prior to release into the fumigation chamber results in higher pest mortality. It is believed vaporisation encourages better mixing, distribution and penetration of the ethyl formate into the produce. However, blackened patches of peel were still observed on much of the fruit suggesting that unacceptable concentration gradients are still present in the fumigation chamber.

It has now been discovered that the methods of the invention allow a high concentration of ethyl formate to be applied without damage to the plant produce occurring.

In the methods of the invention the fumigant may be introduced into the pre-evacuated fumigation chamber or package as a vapour mixture with CO₂ and air. In these embodiments the pesticide concentration in the first and second pesticide mixtures will be substantially the same, corresponding to the desired application rate of the pesticide.

The vapour mixture of fumigant, CO₂ and air can be introduced using any means known in the art. For example, a vapour mixture of VAPORMATE® and air can be introduced by combining a vaporising system (for example, as described above) with a mixer in which known quantities of VAPORMATE® and air are mixed and the pressure equalised. The VAPORMATE®/air mixture is then released through an outlet connected to an application tube. The VAPORMATE®/air ration can be altered by changing the orifice sizes of the inlet nozzles feeding into a pressure equaliser or into a mixing chamber. For example, Table 1 below shows the inlet nozzle sizes that may be used to create a range of VAPORMATE®/air mixtures from a VAPORMATE® source of 5516-5861 kPa (800-850 psi) and a compressed air source of 139 kPa (20 psi), and equalised to atmospheric pressure.

TABLE 1

| VAPORMATE ® | | | | % VAPORMATE ® |
|---|---|---|---|---|
| Orifice | | Compressed Air | | 100 * [#1²/#1² + #2²] (% |
| #1 (mm) | #1² | Orifice #2 (mm) | #2² | by volume) |
| 0.5 | 0.25 | 3.0 | 9.00 | 3% |
| 0.5 | 0.25 | 2.2 | 4.84 | 5% |
| 0.5 | 0.25 | 1.8 | 3.24 | 7% |
| 0.9 | 0.81 | 3.0 | 9.00 | 8% |
| 0.5 | 0.25 | 1.3 | 1.69 | 13% |
| 0.9 | 0.81 | 2.2 | 4.84 | 14% |
| 1.3 | 1.69 | 3.0 | 9.00 | 16% |
| 0.9 | 0.81 | 1.8 | 3.24 | 20% |
| 0.5 | 0.25 | 0.9 | 0.81 | 24% |
| 1.8 | 3.24 | 3.0 | 9.00 | 26% |
| 0.9 | 0.81 | 1.3 | 1.69 | 32% |
| 1.3 | 1.69 | 1.8 | 3.24 | 34% |
| 2.2 | 4.84 | 3.0 | 9.00 | 35% |
| 1.8 | 3.24 | 2.2 | 4.84 | 40% |
| 3.0 | 9.00 | 3.0 | 9.00 | 50% |

As can be seen in Example 3, application of ethyl formate as a vapour mixture with CO₂ and air results in effective fumigation of bananas without blackening of the peel.

Without being bound by theory it is believed that this is because the methods of the invention result in the fumigation chamber or package containing a substantially homogenous mixture of ethyl formate, CO₂ and air. Consequently, the ethyl formate concentration is constant throughout the entire volume being disinfested. This eliminates “hot and cold spots” of high and low ethyl formate concentration, respectively. Plant produce located in a hot spot may be damaged by the relatively higher ethyl formate concentration while plant produce in a cold spot may not be effectively fumigated. Using the conventional method of vaporised VAPORMATE® the effective concentrations of ethyl formate around the volume being disinfested may vary extensively from the average concentration introduced into the chamber or package.

### 6. AEROSOL FORMATION

In one embodiment, the aerosolised pesticide comprises droplets or particles of about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 μm in diameter, and useful ranges may be selected between any of these values (for example, about 2 to 20 μm).

In another embodiment the aerosolised pesticide comprises droplets or particles having an average size of about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 μm in diameter, and useful ranges may be selected between any of these values (for example, about 2 to 20 μm).

In the methods of the present invention the first pesticide mixture is substantially evenly introduced into the air pressure in the upper region of the chamber. The system is then run until the first pesticide mixture has diffused into the lower region of the chamber, forming a substantially homogenous second pesticide mixture throughout the majority of the chamber. This allows a higher concentration of ethyl formate to be used without the risk of damage to the plant material from “hot spots” of much higher concentration. The substantially homogenous higher concentration used ensures that all unwanted organisms exposed to the lethal concentration are eliminated.

The methods of the invention allow pesticides to be applied to plant material in a manner that achieves an acceptable balance of biocidal activity and damage to the produce.

The amount of pesticide required will depend on the volume to be disinfested, target pests and life stages, the amount of plant produce within the volume, sorption potential of the produce leading to loss of lethal concentration in the free space, the temperature of the chamber and the exposure time. In general, a lower concentration of fumigant will require a longer exposure time.

It is to be understood that a skilled worker will be able, without undue experimentation and with regard to that skill and this disclosure, to ascertain the appropriate amount of pesticide required.

In one embodiment the chamber is provided with a fan, to promote rapid dispersion of the pesticide mixture throughout the chamber.

The methods of the invention can be used in conjunction with other disinfestation methods. In one embodiment the method of the invention precedes or follows disinfestation with an alternative pesticide. Examples of alternative pesticides include those listed above.

Methods of the invention can be applied to any of the plant material described above. It may be necessary to first determine the appropriate pesticide to use and appropriate concentrations of pesticide (and optionally CO₂ carrier) for particular plant material and pest combinations. The methods exemplified below may be used by a skilled worker to determine suitable application conditions without requiring undue experimentation.

Various aspects of the invention will now be illustrated in non-limiting ways by reference to the following examples.

### EXAMPLES

### Example 1

Packaged bananas were fumigated using VAPORMATE® (16.7% in 83.3% CO₂ w/w) in aerosol form in a partially pre-evacuated chamber. VAPORMATE® applied through a high pressure gun fitted with nozzle attachment was used to create an aerosol fog. Mites cultured on bean leaves were placed in an insect proof vial consisting of wire netted at the top and bottom to allow the vapour to penetrate. The banana clusters were packaged in one liner, the vial placed under a cluster of bananas allowing the fumigant vapour to reach the pest without an additional barrier.

The results were compared to disinfestation with OFG-1 natural pyrethrins (0.5% pyrethrins, 4.5% ethanol) and sulphur dioxide (pure gas) and an untreated control. Different concentrations of the fumigants were used, measured in g/m³ of fumigant (VAPORMATE®—VM). The concentration of the active ingredient (a.i.) namely ethyl formate—EF used is also given in g/m³ for the VAPORMATE® trial.

Fumigation was carried out at 26° C. for 16 hours against mixed aged long-tailed mealybug (Pseudococcus longispinus) and mixed aged two-spotted mites (Tetranychus urticae). The pests were introduced into the packaged banana boxes in 50 mL insect proof vials and treated.

Pell injury to the fruit was assessed visually as acceptable (−) or unacceptable (+).

### Results:

| | | | % mortality of target pests | | Peel |
|---|---|---|---|---|---|
| Fumigant | (g/m³) | G a.i/m³ | Mealybugs | Mites | injury |
| % VAPORMATE ® | 120 | 20 | 100 | 100 | + |
| (ethyl formate) | 180 | 30 | 99.5 | 100 | + |
| | 240 | 40 | 100 | 99 | + |
| OFG-1 | 600 | 3 | 6 | 19 | − |
| (pyrethrins) | 920 | 5 | 42.5 | 96 | − |
| | 1200 | 6 | 8.5 | 26 | − |
| SO₂ pure | 4 | 0.2 | 5 | 22 | + |
| | 7 | 0.35 | 9 | 30 | + |
| | 10 | 0.50 | 4 | 31 | + |
| Untreated control | | | 3 | 21 | − |

### Example 2

The trial described in Example 1 was repeated using only VAPORMATE® at different concentrations.

Each banana cluster was packed individually with plastic which had several holes to allow for respiration. It was assumed that the pesticide could penetrate and get to the pests. Mites in vials were placed inside the plastic covering the cluster, secured and placed back into the box.

### Results:

| | | | % mortality of target pests | | Peel |
|---|---|---|---|---|---|
| Fumigant | (g/m³) | G EF/m³ | Mealybugs | Mites | injury |
| VAPORMATE ® | 60 | 10 | 100 | 17 | + |
| | 120 | 20 | 100 | 78 | + |
| | 180 | 30 | 100 | 53 | + |
| | 240 | 40 | 100 | 54 | + |
| | 300 | 50 | 100 | 37 | + |

### Example 3

Palletilised packaged bananas were fumigated using vaporised VAPORMATE® (16.7% in 83.3% CO₂ w/w) in a number of partially pre-evacuated chambers.

Target pests: Mixed aged two-spotted mites (Tetranychus urticae). Pests were introduced into the packaged banana boxes in 50 mL insect proof vials and treated.

### Methods of Application:

(A) Single application of:

1. Aerosol using a handgun

2. Product vaporized as it passes through a heated coil using 1 or 5 kW heater.

(B) Multiple doses for the duration of exposure.

Fumigation chambers: (1) GrainPro Cocoon; (2) Boracure custom; (3) Shipping container.

Volume of pallet in relation to total space available was 50-60%.

Duration of exposure: 16 hours for all the single dose applications and 6 hours for the single dose application with top-ups. Temperature 20-26° C. (both trials).

### Results:

| | | | | | |
|---|---|---|---|---|---|
| VM | EF | | Application | | Peel |
| (g/m³) | (g/m³) | Fumigant Chamber | method | % mortality | injury |

Method of Application: (A)

| | | | | | |
|---|---|---|---|---|---|
| 120^{(A)} | 20 | Boracure ® custom | Partial | 53 | + |
| | | | vacuum/aerosol | | |
| 240^{(A)} | 40 | GrainPro ® cocoon | Partial | 39 | + |
| | | | vacuum/aerosol | | |
| 420^{(A)} | 70 | GrainPro ® | Partial | 73 | + |
| | | | vacuum/Product | | |
| | | | vaporise | | |
| 420^{(A)} | 70 | 20′ shipping container | Product vaporised | 76 | + |
| | | | (1 kW) | | |
| 600^{(A)} | 100 | 20′ shipping container | Product vaporised | 76 | + |
| | | | (5 kW) | | |

Method of Application: (B)

| | | | | | |
|---|---|---|---|---|---|
| 20^{(B)} | 3.34 | 20′ shipping container | Product vaporised | 80 | − |
| | | | with H₂O | | |

### Conclusion

^{(A)}Single dose—Uneven distribution and mixing of the fumigant in the chambers, regardless of dose rates, can cause peel injury to bananas and resulted in poor mortality rates.

^{(B)}Single miticidal dose with top-ups maintain concentration in the fumigation system caused no peel injury and increased mortality rate.

### Example 4

Fumigation using vaporized VAPORMATE® mixed with air in pre-evacuated chamber.

Perishable commodity: Packaged bananas prior to export.

Products: VAPORMATE® (16.7% in 83.3% CO₂ w/w).

Target pests: Mixed aged tropical spider mites, mealybugs and soft scale. Mites were introduced into the packaged banana boxes in 50 mL insect proof vials and naturally infested mealybugs and soft scale were used.

Method of Application: A single insecticidal and miticidal dose applied of a homogenized ethyl formate, CO₂ and air vapour into a pre-evacuated box of bananas. Filling time was determined at 12 seconds.

Fumigation chamber: Plastic wrap around banana for export.

Loading: 13 kg of bananas, the available free space in this box was calculated to be about 20 L or 0.02 m³.

Duration of exposure: 16 hours for single dose applications. Banana surface temperature: 26-32° C.

### Results:

| VM | | % VM in | % mortality of target pest | | | Peel |
|---|---|---|---|---|---|---|
| (g/m³) | g EF/m³ | air | Spider mites | Mealybugs | Soft scale | Injury |
| 0 | 0 | 0 | 0.0 | 0.3 | 12.4 | − |
| 162 | 0.84 | 8 | 26.9 | 82.1 | 100 | − |
| 543 | 2.82 | 27 | 96.5 | 100 | 100 | − |
| 825 | 4.30 | 41 | 99.4 | 100 | 100 | − |

### Conclusion

Homogenising a mixture of ethyl formate, carbon dioxide and air before application into the bananas packages allowed for more active ingredient to be applied without causing phytotoxicity to peel.

Preferably, all examples test a range of ethyl formate/VAPORMATE® concentrations (given in standard units) and give pest mortality rates (for at least one species of pest) and fruit damage rates.

### Example 5

A number of trials were then undertaken to determine the efficacy of large-scale delivery of pesticide aerosols using the ENVIROSOL® greenhouse pesticide application system. The ENVIROSOL® system uses a pesticide concentrate comprising a pesticide such as ethyl formate dissolved in liquid carbon dioxide at high pressure. A network of aluminum pipes were located throughout the greenhouse, exiting in clusters of nozzles which aerosolize the pesticide concentrate into the greenhouse. Crop damage was observed in plants in the vicinity of the nozzle clusters. It is though this crop damage is due to the separation of the pesticide concentrate and carbon dioxide caused by the heat differential effects from the flow of the pressurized liquid pesticide concentrate in the aluminum piping resulting in poor aerosol formation with “spluttering” observed from the nozzles.

### Example 5-1

Greenhouse: Dimension 54×37 m², Plastic cladded, Area 2,000 m², Volume 10,000 m³.

Crop: Eggplants for domestic market.

Target pest: Adult whitefly (Trialeurodes vaporaniorum)

Greenhouse pesticide application system: Network of ¼ inch aluminum pipes terminating into four sections of the greenhouse. Each section consisting of a cluster of four standard BOC nozzles to create and distribute the product.

Products sprayed: Permigas @ 2 g/m³.

Frequency of spraying: A total of four sprays at weekly intervals

### Results:

Efficacy: Poor control of whitefly (<50% mortality after each spray)

Phytotoxicity: Severe burns of eggplants leaves were observed at a 2 m radium under each nozzle cluster after each spray. Oil sensitive papers place under the nozzles were stained black showing system produced larger undesirable droplets or limited aerosol production.

### Example 5-2

Greenhouse: Dimension 82×49 m², Plastic cladded, Area 4,000 m², Volume 20,000 m³

Crop: Eggplants for domestic market

Target pest: Adult whitefly (Trialeurodes vaporariorum)

Greenhouse pesticide application system: network of ¼ inch aluminum pipes terminating into four sections of the greenhouse, each section consisting of a cluster of four standard BOC nozzles to create and distribute the product.

Products sprayed: Pestigas @ 0.7 g/m³

Frequency of spraying: A total of four sprays at weekly intervals

### Results:

Efficacy: Poor control of whitefly (<50% mortality after each spray)

Phytotoxicity: Severe burns of eggplants leaves were observed at a 2 m radius under each nozzle cluster after each spray. Oil sensitive papers place under the nozzles were stained black showing system produced larger undesirable droplets or limited aerosol production.

### Example 5-3

Greenhouse: Dimension 23×39 m², Plastic cladded, Area 900 m², Volume 2,700 m³

Crop: Capsicum for domestic market

Target pest: Greenhouse aphids (Myzus persicae)

Greenhouse pesticide application system: Network of ¼ inch aluminum pipes terminating into four standard BOC nozzles located at the corners of the greenhouse to distribute the product.

Products sprayed: ArmourCrop Insecticide [DDVP] @ 1 g/m³

Frequency of spraying: A total of four sprays at weekly intervals

### Results:

Efficacy: Good control of aphid (70% mortality after each spray)

Phytotoxicity: Isolated symptoms of capsicum foliage injury was observed particularly under the nozzles.

### Example 5-4

Greenhouse: Dimension 39×19 m², Plastic cladded, Area 741 m², Volume 2,200 m³

Crop: Cucumbers for domestic market

Target pest: Powdery mildew (Spbaerotheca fuliginea)

Greenhouse pesticide application system: Network of ¼ inch aluminum pipes terminating into four standard BOC nozzles located at the corners of the greenhouse to distribute the product.

Products sprayed: ArmourCrop Fungicide [Mildew] @ 2 g/m³

Frequency of spraying: A total of four sprays at weekly intervals

### Results:

Efficacy: Good control of powdery mildew (<1% plants infected with the disease)

Phytotoxicity: Severe foliage damage observed under each nozzle.

### Example 5-5

Greenhouse: Dimension 49×41 m², Glass, Area 2000 m², Volume 7,000 m³

Crop: Cucumbers for domestic market

Target pest: Powdery mildew (Spbaerotheca fuliginea)

Greenhouse pesticide application system: Network of ¼ inch aluminum pipes terminating into four standard BOC nozzles located in the middle of the greenhouse to distribute the product. Horizontal air circulation fans used to circulate aerosols.

Products sprayed: ArmourCrop Fungicide [Mildew] @ 2 g/m³

Frequency of spraying: A total of four sprays at weekly intervals

### Results:

Efficacy: Good control of powdery mildew (less than 1% plants infected with the disease)

Phytotoxicity: Severe foliage damage observed under each nozzle in the path of spraying.

### Conclusions

Poor-good efficacy was observed in the different trials, however, severe phytotoxicity was observed in all trials in crops within the vicinity of the nozzles after each spray, due to limited or poor aerosol production.

### Example 6

The efficacy of a range of ENVIROSOL® products were then tested by first mixing the aerosolized pesticide with a diluent gas carrier and distributing the pesticide mixture through a duct assisted fan delivery system.

### Example 6-1

Greenhouse: Research, Dimension 16×7 m², Glass, Area 112 m², Volume 315 m³

Crop: Cucumbers

Objective: Phytotoxicity assessment only

Greenhouse pesticide application system: Premixed aerosol/air mixture and duct-assisted distribution.

Products sprayed: ArmourCrop Fungicide [Mildew] @ 2 g/m³

Frequency of spraying: A total of four sprays over 2 days were carried out

### Results:

Phytotoxicity: No systems of foliage injury were observed.

### Example 6-2

Greenhouse: Research, Dimension 16×7 m², Glass, Area 112 m², Volume 315 m³

Crop: Cucumbers

Objective: Phytotoxicity and efficacy assessment

Greenhouse pesticide application system: Premixed aerosol/air mixture and duct-assisted distribution.

Products sprayed: ArmourCrop Insecticide [Methomyl] @ 1 g/m³ plus Permigas @ 2 g/m³

Frequency of spraying: A total of two sprays at 3-days interval

### Results:

Efficacy: >90% mortality of whitefly was observed

Phytotoxicity: No systems of foliage injury were observed.

### Example 6-3

Greenhouse: Dimension 54×37 m², Plastic cladded, Area 2,000 m², Volume 10,000 m³

Crop: Eggplants for domestic market

Target pest: Adult whitefly (Trialeurodes vaporariorum)

Greenhouse pesticide application system: Premixed aerosol/air mixture and assisted-assisted distribution.

Products sprayed: ArmourCrop Insecticide [Methomyl] @ 1 g/m³ plus Permigas @ 2 g/m³

Frequency of spraying: A total of three sprays at 4 days intervals

### Results:

Efficacy: good control of whitefly (>90% mortality after each spray)

Phytotoxicity: No systems of foliage injury were observed.

### Example 6-4

Greenhouse: Research, Dimension 16×7 m², Glass, Area 112 m², Volume 315 m³

Crop: Cucumbers

Objective: Phytotoxicity and efficacy assessment

Greenhouse pesticide application system: Premixed aerosol/air mixture and assisted-assisted distribution.

Products Screened:
ArmourCrop Insecticide [Methomyl] @ 1 g/m³ Permigas @ 2 g/m³
ArmourCrop Insecticide [Methomyl] @ 1 g/m³ plus Armour Crop Insecticide [DDVP] @ 1 g/m³
ArmourCrop Insecticide [Methomyl] @ 1 g/m3 plus Permigas @ 1 g/m3
ArmourCrop Fungicide [Mildew] @ 2 g/m³

Frequency of spraying: Two products were sprayed at a time, changing to a different combination after a 3-days interval

### Results:

Efficacy: >90% mortality of whitefly was observed after each spray

Phytotoxicity: No systems of foliage injury were observed.

### Conclusion

Premixing the aerosolized pesticide, carbon dioxide and air before application into the greenhouse allowed for better distribution of the aerosol throughout the greenhouse, resulting in high levels of efficacy and no damage to plant material.

### INDUSTRIAL APPLICATION

The methods of the invention have application for disinfesting plant material. The methods can be used during growing, before, during or after harvesting, before, during or after packaging, or before, during or after transportation; for example, as a pre-shipment treatment to eliminate quarantine-regulated pests. The methods of the invention can also be used on plant material as part of a sterilization procedure upon arrival at its destination.

Those persons skilled in the art will understand that the above description is provided by way of illustration only and that the invention is not limited thereto.

1-40. (canceled)

41. A method of disinfecting plant material in a chamber, the method comprising:
vaporising pesticide;
mixing the vapour with a volume of a diluent gas to create a first pesticide mixture;
introducing the first pesticide mixture into the chamber; and
circulating the first pesticide mixture throughout a majority of the chamber to form a substantially homogenous second pesticide mixture.

42. The method of claim 41, wherein pressure in the chamber is slightly above atmospheric pressure.

43. The method of claim 41, wherein the diluent gas is air.

44. The method of claim 41, wherein the diluent gas is gas in the chamber.

45. The method of claim 41, comprising recycling the gas in the chamber until the substantially homogeneous second pesticide mixture is formed in the chamber.

46. The method of claim 41, comprising introducing the first pesticide mixture into the chamber using a network of ducting.

47. The method of claim 41, comprising completely vaporizing the pesticide and mixing the pesticide with the diluent gas.

48. The method of claim 41, comprising maintaining the second pesticide within the chamber for a time sufficient to disinfect the plant material.

49. The method of claim 41, wherein the first pesticide mixture has a higher pesticide concentration than the second pesticide mixture.

50. The method of claim 41, further comprising sealing and optionally hermetically sealing the chamber.

51. The method of claim 41, wherein the pesticide comprises a liquid pesticide composite dissolved in liquid CO₂ and stored under pressure.

52. The method of claim 41, comprising heating the pesticide prior to mixture with the diluent gas.

53. The method of claim 1, wherein the first pesticide mixture comprises from about 50% to about 80% of the diluent gas by volume.

54. The method of claim 41, wherein the first pesticide mixture comprises from about 10% to 45% of one or a plurality of pesticides by volume.

55. The method of claim 41, wherein the first pesticide mixture comprises 1-8% ethyl formate, 11-88% CO₂ and 4-88% air by volume.

56. The method of claim 41, comprising maintaining the second pesticide mixture in the chamber for between about 2-48 hours.

57. The method of claim 41, comprising evacuating the chamber prior to the introducing of the first pesticide mixture into the chamber.

58. The method of claim 41, comprising evacuating the chamber simultaneously with the introducing of the first pesticide mixture into the chamber.

59. The method of claim 41, wherein the chamber comprises a vent for displacing gas in the chamber during the introducing the first pesticide mixture.

60. The method of claim 41, wherein the vaporizing comprises aerosolising the pesticide to form droplets each of which has a diameter of from 0.5 to 30 mm.

61. The method of claim 60, wherein the droplets in the first pesticide mixture are from 0.5 to 30 μm in diameter.

62. An apparatus for disinfesting plant material in a chamber, comprising:
a pesticide supply for supplying a vaporising or aerosolised pesticide;
a mixing device for receiving and mixing the pesticide vapour or aerosol with a volume of diluent gas to produce a first pesticide mixture;
a transfer system for introducing the first pesticide mixture into the chamber; and
a circulating system for circulating the first pesticide mixture throughout a majority of the chamber to form a substantially homogenous second pesticide mixture.

63. The apparatus of claim 62, wherein the transfer system comprises ducting to transfer the first pesticide mixture from a mixer to the chamber.

64. The apparatus of claim 62, wherein the transfer system comprises flexible ducting.

65. The apparatus of claim 62, wherein the transfer system comprises flexible ducting having a composition selected from aluminium and plastic.

66. The apparatus of claim 65, wherein the transfer system comprises layflat plastic tubing.

67. The apparatus of claim 62, adapted to disinfest plant material in a greenhouse.

68. The apparatus of claim 62, wherein the transfer system comprises ducting having a diameter of from 100 mm-500 mm.

69. The apparatus of claim 62, wherein the transfer system is adapted to provide the aerosol droplets with a diameter of 0.5 μm to 30 μm to the chamber.

70. The apparatus of claim 62, wherein the transfer system comprises ducting having a plurality of holes spaced along its horizontal axis and optionally above the horizontal axis.

71. The apparatus of claim 70, wherein each of the plurality of holes has a diameter of between 10 and 50 mm.

72. The apparatus of claim 70, wherein the ducting is about 400 mm in diameter, with the holes offset from each other along the horizontal axis of the ducting.

73. The apparatus of claim 62, wherein the pesticide supply comprises a liquid pesticide concentrate dissolving liquid CO₂ and stored under pressure.

74. The apparatus of claim 62, adapted to provide said second pesticide mixture to the chamber such that the chamber is no more than slightly above atmospheric pressure.

75. The apparatus of claim 62, adapted to provide said first pesticide mixture to the chamber such that the chamber is above atmospheric pressure.

76. The apparatus of claim 62, wherein the mixing device is located exterior to the chamber to mix the vaporising or aerosolised pesticide with the diluent gas outside the chamber.

77. The apparatus of claim 62, wherein the mixing device is located within the chamber such that the gas inside the chamber is used as the diluent gas for producing the first pesticide mixture.

78. The apparatus of claim 62, further comprising heating means for heating the first pesticide mixture prior to mixing with the diluent gas.
